(19) United States
(12) Patent Application Publication
(10) Pub. No.: US 2003/0187164 A1
(43) Pub. Date: Oct. 2, 2003

(54) Solutions of lithium pyrrolidine in tetrahydrofuran (thf)/hydrocarbons

(76) Inventors: Wilfried Weiss, Oberursel (DE); Ute Emmel, Frankfurt am Main (DE); Franz Totter, Rodgau (DE); Ulrich Wietelmann, Friedrichsdorf (DE)

Correspondence Address: FULBRIGHT & JAWORSKI, LLP, 666 FIFTH AVE, NEW YORK, NY, 10103-3198, US

(21) Appl. No.: 10/296,519

(22) PCT Filed: May 17, 2001
(86) PCT No.: PCT/EP01/05609

(30) Foreign Application Priority Data
Jun. 7, 2000 (DE) 100 27 604.0

(51) Int. Cl. C08F 4/46; C09K 3/00; C07D 207/00

(52) U.S. Cl. 526/180; 548/400; 252/1

(57) ABSTRACT

The invention relates to solutions of lithium pyrrolidine in a mixture consisting of tetrahydrofuran (THF) and of hydrocarbons, whereby the lithium pyrrolidine concentration ranges from 2 to 30 wt. %, and the molar ratio Li pyrrolidine: THF=1: 0.5 to 1: 1.5. The invention also relates to methods for preparing these solutions and to uses of these solutions.

The invention relates to solutions of lithium pyrrolidine in mixtures of THF and hydrocarbons, and also to methods for their preparation and to their use.

Lithium pyrrolidine belongs to the family of lithium organoamides. These are reagents that are used in a versatile manner in organic synthesis and are distinguished by a high basicity with relatively low nucleophilicity.

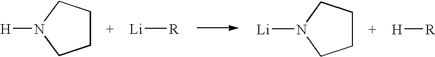

The reaction can generally be effected in ethers or hydrocarbons, with, for example, methyl lithium being used in ether or n-butyl lithium being used in hexane (Houben-Weyl, “Methoden der Organischen Chemie”, published by Thieme Verlag, Volume XIII/1, 1970; Wakefield, B. J. “The Chemistry of Organolithium Compounds”, Pergamon Press, London, 1974; “Organolithium Methods”, Academic Press, London, 1988; Brandsma, L. “Preparative Polar Organometallic Chemistry”, Vol 1, 1987, and Vol 2, 1990, published by Springer-Verlag; M. Schlosser et al, “Organometallics in Synthesis”, 1994, John Wiley & Sons, Sussex).

A further possibility for the synthesis of Li-organoamides lies in the Ziegler method of reacting the amine with lithium in the presence of a diene:

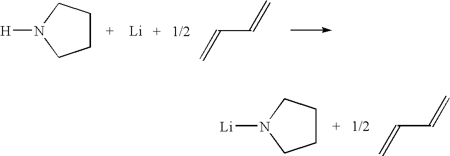

(K. Ziegler, et al., Liebigs Ann. Chem. 511 64, 1934; Reetz. M. T., et al., Liebigs Ann. Chem., 1471, 1980; WO-97/21714).

Li-organoamides are usually solid substances with pyrophoric properties.

In order that they can he handled more easily, above all in the industrial field, they are marketed in solution; hydrocarbon/ether mixtures with various additives which have a positive influence upon the solubility and stability are used as a solvent.

The solubility in hydrocarbons is often too low so that additives, such as Li-alkoxides, are used (DE 4332652.8).

The solubility in ethers is generally good, though these solutions decompose at room temperature, which is why they are not commercially available (Leo. A. Paquette, “Encyclopaedia of Reagents for Organic Synthesis”, Vol. 5, 3163, John Wiley, 1995).

In order to reduce the decomposability of the ethereal solutions, for example in the case of the lithium-di(i-propyl)amide (LDA), a solvent mixture of THF and hydrocarbon is used in which the THF is limited to a content≦1.0 mol/mol LDA (WO-86/03 744).

It is also possible to add stabilizers, such as Li-halides (U.S. Pat. No. 5,679,850).

A further possibility of stabilizing LDA, for example, lies in the addition of magnesium-bis(organo)amides (DE 3905857, U.S. Pat. No. 5,320,774), although this gives rise to the disadvantage of changed reactivity as a result of the addition of the alkaline earth metal.

A paper by Nudelmann, N. S. et al. gives information on the special properties of Li-pyrrolidine (J. Chem. Soc. Perkin Trans. 2 (1990), (8), 1461-5); its specific use in pharmasynthesis is also known (Simvastatin Synthesis: U.S. Pat. No. 5,223,415, Merck 1992).

In solid form Li-pyrrolidine is present as a white, amorphous material with a polymeric conductor structure which is insoluble in hydrocarbons. The crystal structures with the Lewis bases PMDETA (pentamethyl diethylenetriamine) and TMEDA (tetramethyl ethylenediamine) are known (R. Snaith et al., J. Chem. Soc., Chem. Commun., 1986 869; R. Snaith, JACS; 1989, 111,4719).

Since lithium pyrrolidine, on account of its special chemical properties, clearly contrasts with LDA and is of particular interest for organic synthesis, stable, non-pyrophoric commercial forms are desirable, although these have not been known hitherto. Lithium pyrrolidine occupies a special position within lithium organoamides; on account of its strong basicity it is particularly reactive and attacks THF much more intensely than, for example, LDA. An object of the invention is therefore to provide sufficiently concentrated, stable and non-pyrophoric solutions of lithium pyrrolidine.

The object is achieved by means of solutions of lithium pyrrolidine in a mixture of tetrahydrofuran (THF) and hydrocarbons, with the lithium pyrrolidine concentration amounting to 2 to 30% by weight, preferably 5 to 25% by weight, and with the molar ratio of Li-pyrrolidine: THF=1:0.5 to 1:1.5.

Although in the Encyclopaedia of Reagents for Organic Syntheses, Vol. 5, published by L. A. Paquette, it is disclosed on page 3163 that solutions of lithium pyrrolidine in THF are unstable at room temperature and form LiH after a short time, it has been found that lithium pyrrolidine in the molar ratio to THF in accordance with the invention has a quite special complexing behaviour and that this completing behaviour determines both the solubility in hydrocarbons and the stability of these solutions with regard to crystallization when cooling and with regard to thermal decomposition when heating. On the basis of IR-spectroscopic findings and calorimetric data, it has been possible to show that precisely in the concentration range in accordance with the invention the attack of lithium pyrrolidine on THF does not occur to a large extent, the THF-complexing is promoted and the complexing effects a positive influence upon the stability and the solubility. Furthermore, it has been shown that the complexes in accordance with the invention are dissolved in hydrocarbons with an increasing molar proportion of THF (see Table 1).

TABLE 1
Solubility of lithium pyrrolidine in various
THF/hydrocarbon mixtures (numerical data in %
by weight Li-Py)

| | Li-Py: THF | Li-Py: THF |
|---|---|---|
| | ratio | ratio |
| Hydrocarbon | 1: 0.5 | 1: 1.5 |
| Hexane | 7% | 18% |
| Cyclohexane | 12% | 23% |
| Toluene | 13% | 24% |

Furthermore, it has been shown that the solutions in this range are also sufficiently stable in storage. Thus a 17% solution of Li-Py in THF (without hydrocarbon) has a decomposition rate of k=−0.9% Li-Py/day at 0° C. and k=−4.5% Li-Py/day at 26° C. In contrast thereto, a 17% solution of the composition 1 Li-Py: 1.2 THF in toluene in the range of 0° C. to +40° C. proves to be distinctly more stable (see Table 2).

TABLE 2
Decomposition rates of 17% Li-Py solution in
THF/toluene with a Li-Py/THF ratio of 1: 1.2

| | Decomposition rate |
|---|---|
| | Li-Py: THF 1: 1.2 in |
| Temperature | toluene |
| 0° C. | k= stable |
| 20° C. | k= stable |
| 40° C. | k= −1% Li-Py per day |

A decomposition reaction of the 1st order has been assumed for the determination of the stability; for this the following holds good:

k=ln [final concentration_{(active base)}/initial concentration_{(active base)}]×100/storage time in days.

The active base was determined according to a modified Watson-Eastham Method (S. C. Watson, J. F. Eastham, J. Organomet. Chem. 9, 165, 1967; L. Duhamel, J. C. Plaquevant, JOC, 44, 3404, 1979). The ratio of Li-Py: THF was determined by means of 1-H-NMR spectroscopy.

The solutions in accordance with the invention of Li-pyrrolidine in a mixture of THF and hydrocarbons with the mixing ratio specified exhibit such good dissolving and stability behaviour that they are suitable for industrial use.

The synthesis of the solutions in accordance with the invention can be effected in different ways:

On the one hand, metallic lithium can be reacted according to the Ziegler method in the presence of corresponding quantities of pyrrolidine and THF in a hydrocarbon with a diene. The lithium can then be used as a powder or granulated material in hydrocarbon. The quantities of Li, pyrrolidine, THF and hydrocarbon used are determined by the desired Li-pyrrolidine concentration and by the desired Li-pyrrolidine-THF ratio of the solution obtained. This ratio in turn influences the desired solubility and stability. Isoprene is preferably used as the diene, although the use of styrene and other dienes is also possible. The reaction is exothermal; in order to avoid thermal decomposition, operation is preferably carried out at 0° C. to 20° C., with it being possible to regulate the heat by way of the dosing rate.

On the other hand, however, the pyrrolidine can also be reacted with a lithium-organyl, such as n-butyl lithium, in a hydrocarbon to form the Li-pyrrolidine. The Li-pyrrolidine precipitated from this solution is then dissolved with a quantity of THF, corresponding to the desired ratio of Li-pyrrolidine to THF, in the hydrocarbon.

The solutions in accordance with the invention are used in organic-chemical synthesis and as a polymerization catalyst.

The invention is explained in greater detail in the following with the aid of examples.

### EXAMPLE 1

10.4 g (1.5 mol) Li-granulate were placed in 90.1 g (1.25 mol) THF and 71.1 g (1 mol) pyrrolidine at 20° C. A mixture of 34.1 g (0.5 mol) isoprene and 200 ml toluene was added to this in uniform metered doses within 2 hours, and the resulting heat was dissipated by jacket-cooling. After the reaction had died out, excess Li-metal was filtered off. 380 g solution was obtained with an active base content of 16.6% Li-pyrrolidine, this corresponding to a yield of 82%.

### EXAMPLE 2

10.4 g (1.5 mol) Li-granulate were placed in 90.1 g (1.25 mol) THF and 71.1 g (1 mol) pyrrolidine in 200 ml cyclohexane at 20° C. 34.1 g (0.5 mol) isoprene were added thereto in metered doses within 3 hours. After a 1 hour secondary reaction, the solution was filtered. 350 g solution were obtained with an active base content of 17.1%, this corresponding to a yield of 78%.

### EXAMPLE 3

8 g (1.15 mol) Li-granulate were placed with 73.8 g (1.3 mol) THF and 71.1 g (1 mol) pyrrolidine in 850 ml hexane at 20° C. and reacted with 34.1 g (0.5 mol) isoprene within 3 hours. After a 1 hour secondary reaction, the solution was filtered. 730 g clear solution were obtained with an active base content of 8.5%, this corresponding to a yield of 80%. Upon cooling, 61 g crystals were precipitated from this solution, the composition of the crystals being determined with 1 Li-Py×0.6 THF.

The solubility of the crystals that resulted was 7% by weight in hexane, 14.4% by weight in cyclohexane, and 13.5% by weight in toluene. By adding THF up to the ratio of 1 Li-Py:1.5 THF, the solubility increased to 18.8% by weight in hexane, to 23.5% by weight in cyclohexane and to 24.0% by weight in toluene.

### EXAMPLE 4

90 g of an 18% n-butyl lithium solution in toluene (corresponds to 250 mmol n-BuLi) were reacted with 17.8 g (corresponds to 250 mmol) pyrrolidine within 30 minutes at room temperature with counter-cooling; a colourless deposit was formed. By adding 21.6 g (300 mmol) THF, the Li-pyrrolidine that was formed was dissolved. A 17.1% solution of Li-pyrrolidine (2.22 mmol active base per g solution) was obtained in THF/toluene with a molar Li-pyrrolidine/THF ratio of 1:1.2. A stability test was carried out with this solution in a closed glass vessel in a tempered oil bath (Example 5).

### EXAMPLE 5

Stability Test

The solution from Example 4 was tested for its stability.

TABLE 3
Change in the active base after 16 days
storage time at various temperatures

| Storage time/ | Temperature | Active |
|---|---|---|
| days | 0° C. | base/mmol/g |
| 0 | | 2.22 |
| 16 | 0 | 2.22 |
| 16 | 20 | 2.21 |
| 16 | 40 | 1.91 |

Comparative Example: Stability of lithium pyrrolidine in THF without hydrocarbon

6.9 g (1 mol) Li-powder were reacted with 34.1 g (0.5 mol) isoprene in 300 ml THF and 71.1 g (1 mol) pyrrolidine at 20° C. within 3 hours and filtering took place after a 1 hour secondary reaction. 325 g solution were obtained with an active base content of 17.1% (yield 82%). After storage for 14 days at 20° C. this solution only had an active base content of 1.70 mmol/g.

1. Solutions of lithium pyrrolidine in a mixture of tetrahydrofuran (THF) and hydrocarbons, wherein the lithium pyrrolidine concentration amounts to 2 to 30% by weight and wherein the molar ratio of Li-pyrrolidine:THF=1:05 to 1:15.

2. Solutions according to claim 1, characterised in that the lithium pyrrolidine concentration amounts to 5 to 25% by weight.

3. Solutions according to claim 1 or 2, characterised in that aliphatic hydrocarbons (cyclic or acyclic) with 5 to 12 C-atoms or aromatic hydrocarbons with 6 to 12 C-atoms are used as the hydrocarbons.

4. Solutions according to claim 3, characterised in that one or more of the compounds pentane, hexane, heptane, octane, cyclohexane, tetralin, toluene, xylene, cumene or ethyl benzene are used as the hydrocarbons.

5. Method for preparing a solution according to claims 1 to 4, characterised in that metallic lithium is reacted in the presence of pyrrolidine and THF in a hydrocarbon with a diene.

6. Method according to claim 5, characterised in that the lithium is used as a powder or as a granulate.

7. Method according to one of claims 5 or 6, characterised in that butadiene or one of its derivatives or styrene or naphthalene is used as the diene.

8. Method according to claim 7, characterised in that isoprene is used as the diene.

9. Method for preparing a solution according to claims 1 to 4, characterised in that pyrrolidine is reacted with a lithium-organyl in a hydrocarbon to for Li-pyrrolidine, and the Li-pyrrolidine precipitated from this solution is dissolved with a quantity of THF, corresponding to the desired ratio of Li-pyrrolidine to THF, in the hydrocarbon.

10. Method according to one of claims 5 to 9, characterised in that the temperature of the reaction is preferably held at 0° to 20° C.

11. Use of the solutions according to claims 1 to 4 for organic-chemical synthesis and as a polymerization catalyst.
